(19) United States
(12) Patent Application Publication
(10) Pub. No.: US 2006/0287273 A1
(43) Pub. Date: Dec. 21, 2006

(54) Methods and reagents for inhibiting cell proliferation

(76) Inventors: Donald J. Fujita, Calgary (CA); Jeffrey D. Bjorge, Calgary (CA)

Correspondence Address: BIRCH STEWART KOLASCH & BIRCH, PO BOX 747, FALLS CHURCH, VA, 22040-0747, US

(21) Appl. No.: 11/453,099

(22) Filed: Jun. 15, 2006

Related U.S. Application Data
(60) Provisional application No. 60/690,876, filed on Jun. 16, 2005.

(51) Int. Cl. A61K 48/00; C07H 21/02

(52) U.S. Cl. 514/44; 536/23.1

(57) ABSTRACT

Methods and reagents for reducing expression of proteins involved in intracellular signalling are disclosed. Specifically, siRNA sequences are provided for reducing expression of Src, stat3, and c-myc gene products. Combination treatments are disclosed for inhibiting cell proliferative pathways, and for reducing tumor growth in breast and colon cancer.

### CROSS REFERENCE TO RELATED APPLICATION

This application is based upon and claims the benefit of priority from prior U.S. application Ser. No. 60/690,876, filed Jun. 16, 2005, the entire contents of which are incorporated herein by reference.

### SEQUENCE LISTING

This application includes a Sequence Listing consisting of 11 pages with 26 sequences attached.

### FIELD OF THE INVENTION

The present invention relates generally to methods and reagents for inhibition of cell proliferation. More particularly, the present invention relates to the use of RNAi for silencing certain genes involved in signal transduction pathways related to cancer cell proliferation.

### BACKGROUND OF THE INVENTION

Abnormal cell division contributing to the development of cancer results from the activation of proto-oncogenes or oncogenes, or the suppression or inactivation of tumor suppressor genes, leading to uncontrolled cellular proliferation. With advances in molecular research, tissue-specific gene and protein-targeted therapies are now entering the therapeutic product pipeline and may be used in an attempt to correct or reverse the pathology of certain cancers. Some of the most promising current therapeutic approaches in cancer are those which have been designed to target specific cancers, or subsets of cancers (e.g. Herceptin for breast cancers overexpressing Her-2/Neu receptors, and Imatinib (STI571) against Bcr/Abl, for use in patients with chronic myeloid leukemia and gastrointestinal stromal tumors).

Several intracellular signaling pathways have been implicated in the development of different cancers, and advanced research into these signaling pathways is revealing a certain amount of convergence or interrelationship between these pathways. The extent of such convergence or interdependence remains unclear.

For example, the cytoplasmic and perinuclear kinase Src, is activated in a large fraction of breast cancers. Src is a non-receptor tyrosine kinase that can cause cellular transformation in cell culture, and tumor formation in animals if its activity becomes highly elevated by mutation or dysregulation. Low levels of Src activity are normally present in most cell types, although Src knockout mice have been shown to be viable (Soriano et al, 1991, Cell, Vol. 64, pp. 693-702).

Elevated levels of Src activity have been found in a number of types of human cancers, including cancers of the colon, breast, and ovary (Cartwright et al, 1989, J. Clin. Invest., Vol. 83, pp. 2025-2033; Cartwright et al, 1990, Proc. Natl. Acad. Sci. USA, Vol. 87, pp. 558-562; Egan et al, 1999, Oncogene, Vol. 18, pp. 1227-1237; Irby et al, 1999, Nat. Genet., Vol. 21, pp. 187-190; Jacobs and Rubsamen, 1983, Cancer Res., Vol. 43, pp. 1696-1702; Luttrell et al, 1994, Proc. Nat. Acad. Sci., Vol. 91, pp. 83-87; Muthuswamy et al, 1994, Mol. Cell. Biol., Vol. 14, pp. 735-743; Ottenhoff-Kalff et al, 1992, Cancer Res., Vol. 52, pp. 4773-4778; Rosen et al, 1986, J. Biol. Chem., Vol. 261, pp. 13754-13759; Talamonti et al, 1993, J. Clin. Invest., Vol. 91, pp. 53-60; Bjorge et al, 2000, J. Biol. Chem. Vol. 275, pp. 41439-41446) suggesting it may play an important role in the progression and/or development of these cancers. Src kinase is a central cellular regulatory protein that is capable of activating numerous further signaling pathways, including several that lead to stimulation of proliferation, motility, angiogenesis, and metastasis, and to blockage of apoptosis. As a result, and in contrast to oncogenes such as BCR/ABL, Src's contribution to the phenotype of malignant cells is less well defined, and Src is likely only one of several gene products whose function is abnormal in malignant cells. For example, both Src and its downstream signaling molecules Stat3 and Myc have been implicated in the development, maintenance and/or progression of human cancers, possibly through interdependent signalling pathways (Berclaz et al 2001, Int. J. Oncol. Vol 19, pp. 1155-1160; Wang et al, 2000, Oncogene, Vol. 19, pp. 2075-2085; Zhang et al, J. Biol. Chem, Vol. 175, pp. 24935-24944; Ling and Arlinghaus 2005 Cancer Res., Vol. 65, pp. 2532-2536; Wang et al, 2005, Breast Cancer Res. Vol. 7, pp. R220-228). It is not known which of these targets, if any will be the most effective in preventing cancer progression.

Four activated oncogenic mutant forms of the human Src protein that are capable of transforming cells in culture and causing tumor formation in experimental animals (chickens) have been described and characterized by the Applicants and co-workers. (Tanaka and Fujita, 1986, Mol. Cell. Biol., Vol. 6(11), pp. 3900-3999; Tanaka et al, 1990, Oncogene Res., Vol. 5(4), pp. 305-322; Bjorge et al, 1995, J. Biol. Chem., Vol. 270(41), pp. 24222-24228). A specific oncogenic mutant form of Src containing a nonsense chain-terminating mutation in codon 531 of the human Src protein has been reported to occur in some advanced colon carcinomas (Irby et al, 1999). In addition, the present inventors have found that activated mutant and overexpressed wild-type forms of Src are capable of transforming immortalized human mammary epthelial cells in culture, though tests involving anchorage-independent growth (Achari, Quong, Stampfer and, Fujita, unpublished results.) These findings, collectively, provide evidence that activated forms of Src and/or upregulation of Src may have a role in cell transformation and cancer development and/or malignant progression in experimental animals and humans.

With respect to human breast cancer, overexpression of the tyrosine kinase receptor HER2/neu has been reported in about 20-25% of breast cancer, and mutations in the tumor suppressors BRCA1 or BRCA2 have been implicated in a smaller subset of breast cancers (less than 3%). It is the current-held belief that human breast cancer is the result of multiple cellular events that have affected the growth properties of breast epithelial cells and how they interact with their environment. In addition to the above well-characterized changes, other alterations are less well defined and include activation of the non-receptor tyrosine kinase Src which has been shown in up to 30-70+% of breast cancers by the present inventors and others (Egan et al, 1999; Jacobs and Rubsamen, 1983; Ottenhoff-Kalff, 1992; Rosen et al, 1986). It is not clear whether or how Src activation may be related to the overexpression of HER2/neu and the function of BRCA1 or BRCA2.

Previous studies by the inventors have identified at least five breast cancer cell lines that exhibit a substantial elevation (ca. 3 to 20-fold) of Src kinase activity (Egan et al, Oncogene, 1999). Some of these do not contain overexpressed Her-2/Neu or EGFRs, and the role of Src kinase has not yet been determined in these particular cell lines. Current methods of inhibiting Src/Src kinase have not provided precise results attributable to Src kinase alone, and may not be suitable candidates for therapeutic use, as they produce unwanted significant inhibitory effects on other closely related Src-family members such as the tyrosine kinases fyn, yes, and lck.

In order to specifically study the potential role of various genes in specific cancer cell lines, individual genes may be knocked-down, or silenced using various techniques generally known in the art. RNA interference (RNAi) is a powerful technique which selectively reduces the level of the targeted protein without directly altering the genetic material. RNA is administered as double-stranded RNA, which is engineered to substantially match the target protein mRNA. The cell's natural RNA-induced silencing complex recognizes the siRNA sequence, and targets the matching mRNA for degradation, thereby reducing expression of the target protein. RNAi is generally anticipated to provide a more precise and effective means of interfering with individual protein expression than antisense methods, of which many failed therapeutic attempts abound in the prior art.

The use of RNAi to suppress the expression of a specific mRNA and its encoded protein, followed by the examination of the phenotypic consequences, has proven to be very useful and effective in studies ranging from investigations on viral replication (Capodici et al, 2002, J. Immunol., Vol. 169, pp. 5196-5201; Gitlin et al, 2002, Nature, Vol. 418, pp. 430-434; Jacque et al, 2002, Nature, Vol. 418, pp. 435-438; Jiang and Milner, 2002, Oncogene, Vol. 21, pp. 6041-6048; Novina et al, 2002, Nat. Med., Vol. 8, pp. 681-686) to studies of cytoskeletal proteins (Harborth et al, 2001, J. Cell. Sci., Vol. 114, pp. 4557-4565).

Recently, siRNA targeted against the oncogenes K-RAS^{V12} and BCR/ABL, and Twist, a transcription factor thought to play a key role in tumor metastasis (Yang et al, 2004, Cell, Vol. 117, pp. 927-939), have been shown to suppress the neoplastic phenotype of various types of cancer cells. In the first two cases, the presence of specific mutations allowed design of the siRNA such that it would target only the oncogenic form of these gene products. Under these conditions, siRNA expressed by a retroviral vector to target K-RAS^{V12} was shown to suppress the mutated K-RAS^{V12} oncogene in the human pancreatic cancer cell line CAPAN-1, resulting in suppression of its neoplastic phenotype (Brummelkamp et al, 2002, Cancer Cell, Vol. 2, pp. 243-247). Similarly, direct application by transfection of an siRNA overlapping and targeting the “breakpoint” region of the BCR/ABL mRNA was effective in inducing apoptotic cell death in the human chronic myeloid leukemia cell line K562 (Wilda et al, 2002, Oncogene, Vol. 21, pp. 5716-5724). In the latter case, lentivirus-expressed short hairpin RNA was used to target the normal gene product Twist, that is overexpressed in some invasive types of breast cancer (Yang et al, 2004). This resulted in an inhibition of the metastatic properties of a mouse mammary tumor cell line 4T1.

RNAi treatment has recently been administered to human subjects for the treatment of age-related macular degeneration, an eye disease that destroys central vision by damaging the macula, the central region of the retina. In November 2004, the first clinical study was initiated, involving a chemically optimized siRNA. The Phase I, open-label, dose-escalation trial is testing siRNA in the treatment of patients with a form of age-related macular degeneration. The specific siRNA under investigation in the study is a chemically modified short interfering RNA (siRNA) targeting Vascular Endothelial Growth Factor Receptor-1 (http://www.sirna.com/simaiproduct/sirna-027.html).

There is a need for further reagents useful in the study of cancer cell biology. Moreover, it remains to be determined whether a role exists for siRNA therapeutics in the treatment of cancer.

### BRIEF DESCRIPTION OF THE DRAWINGS

Embodiments of the present invention will now be described, by way of example only, with reference to the attached Figures, wherein:

FIG. 1A is a listing of siRNA sequences for reducing protein expression of Src, stat3, and cmyc;

FIG. 1B is a listing of candidate RNA sequences corresponding to the DNA sequences identified in FIG. 1;

FIG. 2 is a schematic diagram proposing how Src signal transduction may cascade to other signalling pathways as it relates to the invention and data disclosed herein;

FIG. 3a is a SDS-PAGE transferred to nitrocellulose and probed with anti-Src antibody, showing Src expression in untreated MDA-MB-435S cells compared with MDA-MB-435S cells transfected with scrambled or Src-targeted siRNA;

FIG. 3b is a graphical illustration depicting the quantitative analysis of the results shown in FIG. 3a;

FIGS. 4a and b are graphical illustrations of observed MDA-MB-435S cell growth in soft agar and monolayer culture following transfection with reagents as indicated;

FIG. 5a is an immunoassay showing the reduction in Src, MAPK1, and Akt1 protein expression following transfection of MDA-MB-435S cells with specific siRNAs;

FIG. 5b is a graphical representation of siRNA-transfected MDA-MB-435S cell growth on soft agar;

FIG. 5c is a graphical representation of siRNA-transfected MDA-MB-435S cells grown in monolayer culture;

FIG. 6A is a graphical representation of tumor weight in NOD/SCID mice implanted with MDA-MB-435S cells with and without siRNA treatment as shown;

FIG. 6B is a western blot with anti-GFP antibody with respect to the experimental results shown in FIG. 6A;

FIG. 7 is a graphical representation of the results of a TUNEL assay, analyzed by FACS, illustrating the effect of Src siRNA on apoptosis of MDA-MB-435S cells;

FIG. 8 is a graphical representation showing tumor size in mice injected with MDA-MB-435 breast cancer cells following siRNA treatment;

FIG. 9 is a graphical representation showing tumor weight in mice injected with MDA-MB-435 breast cancer cells following siRNA treatment;

FIG. 10a is a graphical representation showing tumor volume in mice injected with SW480 cells (colon cancer) following siRNA treatment by direct injection into tumors;

FIG. 10b is a graphical representation showing tumor volume in mice injected with SW480 cells that were previously transfected with Src and stat siRNA

### DESCRIPTION OF THE INVENTION

In accordance with an aspect of the invention, there is provided an isolated RNA molecule comprising a nucleic acid molecule hybridized to a second nucleic acid molecule, the first nucleic acid molecule selected from the group consisting of:

| | |
|---|---|
| 5′-UUCGGAGGCUUCAACUCCU-3′; | (SEQ. ID. NO. 29) |
| 3′-AAGCCUCCGAAGUUGAGGA-5′; | (SEQ. ID. NO. 30) |
| 5′-GAGAACCUGGUGUGCAAAG-3′; | (SEQ. ID. NO. 31) |
| 3′-CUCUUGGACCACACGUUUC-5′; | (SEQ. ID. NO. 32) |
| 5′-GAAUCAGGCCUUCUACAGA-3′; | (SEQ. ID. NO. 33) |
| 3′-CUUAGUGCGGAAGAUGUCU-5′; | (SEQ. ID. NO. 34) |
| 5′-GGCGUCCAGUUCACUAGUA-3′; | (SEQ. ID. NO. 35) |
| 3′-CCGCAGGUCAAGUGAUGAU-5′; | (SEQ. ID. NO. 36) |
| 5′-GCUUCACCAACAGGAACUA-3′; | (SEQ. ID. NO. 37) |
| 3′-CGAAGUGGUUGUCCUUGAU-5′; | (SEQ. ID. NO. 38) |
| 5′-AAACAUGAUCAUCGAGGAC-3′; | (SEQ. ID. NO. 39) |
| 3′-UUUGUAGUAGUAGGUCCUG-5′; | (SEQ. ID. NO. 40) |
| 5′-AAUGACCUAUGCCUGAGCA-3′; | (SEQ. ID. NO. 41) |
| 3′-UUAGUGGAUACGGACUCGU-5′; | (SEQ. ID. NO. 42) |
| 5′-AACGAUUCCUUCUAACAGA-3′; | (SEQ. ID. NO. 43) |
| 3′-UUGCUAAGGAAGAUUGUCU-5′; | (SEQ. ID. NO. 44) |
| 5′-ACGACGAGACCUUCAUCAA-3′; | (SEQ. ID. NO. 45) |
| and | |
| 3′-UGCUGCUCUGGAAGUAGUU-5′. | (SEQ. ID. NO. 46) |

In one embodiment, the isolated RNA molecule further comprises additional nucleotides, functional groups, antibodies, or other ligands appended directly or indirectly to the RNA molecule. The isolated RNA molecules may be used to reduce cellular proliferation in vivo, and/or to inhibit at least one intracellular signalling pathway.

In accordance with a second aspect of the invention, there is provided an siRNA construct for reducing expression of a protein involved in cellular proliferative signalling, the siRNA construct selected from the group consisting of:

| | |
|---|---|
| 5′-UUCGGAGGCUUCAACUCCUdTdT-3′ | (SEQ. ID. NO. 2) |
| hybridized to | |
| 3′-dTdTAAGCCUCCGAAGUUGAGGA-5′; | (SEQ. ID. NO. 3) |
| 5′-GAGAACCUGGUGUGCAAAGUU-3′ | (SEQ. ID. NO. 5) |
| hybridized to | |
| 3′-UUCUCUUGGACCACACGUUUC-5′; | (SEQ. ID. NO. 6) |
| 5′-GAAUCACGCCUUCUAGAGAUU-3′ | (SEQ. ID. NO. 8) |
| hybridized to | |
| 3′-UUCUUAGUGCGGAAGAUGUCU-5′; | (SEQ. ID. NO. 9) |
| 5′-GGCGUCCAGUUCACUACUAUU-3′ | (SEQ. ID. NO. 11) |
| hybridized to | |
| 3′-UUCCGCAGGUCAAGUGAUGAU-5′; | (SEQ. ID. NO. 12) |
| 5′-GGUUCACCAACAGGAACUAUU-3′ | (SEQ. ID. NO. 15) |
| hybridized to | |
| 3′-UUCGAAGUGGUUGUCCUUGAU-5′; | (SEQ. ID. NO. 16) |
| 5′-AAACAUCAUCAUCCAGGACUU-3′ | (SEQ. ID. NO. 18) |
| hybridized to | |
| 3′-UUUUUGUAGUAGUAGGUGCUG-5′; | (SEQ. ID. NO. 19) |
| 5′-AAUCACCUAUGCCUGAGGAUU-3′ | (SEQ. ID. NO. 21) |
| hybridized to | |
| 3′-UUUUAGUGGAUACGGAGUGGU-5′; | (SEQ. ID. NO. 22) |
| 5′-AACGAUUCCUUCUAACAGAUU-3′ | (SEQ. ID. NO. 24) |
| hybridized to | |
| 3′-UUUUGCUAAGGAAGAUUGUCU-5′; | (SEQ. ID. NO. 25) |
| and | |
| 5′-ACGACGAGACGUUCAUCAAUU-3′ | (SEQ. ID. NO. 27) |
| hybridized to | |
| 3′-UUUGCUGGUCUGGAAGUAGUU-5′. | (SEQ. ID. NO. 28) |

In an embodiment, one or both strands of the siRNA construct further comprise(s) additional nucleotides, functional groups, antibodies, or other ligands appended directly or indirectly to the RNA strand(s).

In accordance with a further aspect of the invention, there is provided a double-stranded RNA molecule for inhibiting Src expression in a cell, wherein one of the RNA strands is homologous to at least a portion of a Src DNA or RNA molecule. In one embodiment, the DNA or RNA molecule is a conserved portion of the Src DNA or RNA molecule. In a further embodiment of this aspect of the invention, the RNA molecule is between 18-23 base pairs in length. The double-stranded RNA molecule may be a hairpin RNA sequence (ie. a single stranded RNA containing a targeting region and a complementary region complementary to the target region such that the single strand may be folded into a double stranded RNA with a hairpin loop) and/or may further comprise additional nucleotides, functional groups, antibodies, or other ligands appended to the RNA molecule.

In a further embodiment of this aspect, the RNA molecule may be used to reduce cellular proliferation in vivo and/or to inhibit at least one intracellular signalling pathway. Still further, the RNA molecule may be so used in the treatment of cancer. In a further embodiment, the cancer may be breast or colon cancer.

In accordance with a further aspect of the invention, there is provided a method to reduce cell proliferation comprising administering to a cell an effective amount of a reagent capable of reducing Src expression; and administering to a cell an effective amount of an additional reagent capable of reducing activity of at least one protein involved in intracellular proliferative signalling. In accordance with an embodiment of this aspect of the invention, the reagent capable of reducing Src expression may be a double stranded RNA molecule. In accordance with a further embodiment of this aspect of the invention, the additional reagent may be a double stranded RNA molecule, an antibody, or a targeted drug therapy reagent, and may be intended to reduce the activity of Src, Stat3, or cMyc.

In accordance with a further aspect of the invention, there is provided a method for cancer treatment comprising administering to a patient an effective amount of a reagent capable of reducing Src expression; and administering to the patient an effective amount of an additional reagent capable of reducing activity of at least one protein involved in intracellular proliferative signalling. In accordance with an embodiment of this aspect of the invention, the reagent capable of reducing Src expression may be a double stranded RNA molecule. In accordance with a further embodiment of this aspect of the invention, the additional reagent may be a double stranded RNA molecule, an antibody, or a targeted drug therapy reagent, and may be intended to reduce the activity of Src, Stat3, or cMyc.

Other aspects and features of the present invention will become apparent to those of ordinary skill in the art upon review of the present description with the accompanying figures.

Generally, the present invention provides a method for inhibiting cell proliferation using RNA interference. siRNA sequences for reducing expression of Src, Stat3, and cMyc, are provided, and combined therapy is shown to have an unexpected additive or synergistic effect in inhibiting proliferation of breast cancer and colon cancer cells and tumors. In particular, the combined use of two or more siRNAs directed to Src and upstream and/or downstream signalling elements inhibits the neoplastic properties of certain human breast cancer and colon cancer cell lines. In mouse studies, administration of siRNAs by transfection with lipid vesicles into cells or administration by direct injection of siRNA into tumors, inhibits tumor growth.

siRNA molecules can be constructed and assembled in accordance with methods generally known in the art. See for example: Wincott et al, 1995 Nucleic Acids Research, Vol. 23, pp. 2677-2684 and 1997, Methods in Molecular Biology, Vol 74, pp. 59; Brennan et al, 1998, Biotechnol. Bioeng., Vol. 61, pp. 33-45; and Sohail et al, Nucleic Acids Res, Vol. 31, No. 7, e38. Alternatively, assembled double-stranded siRNA may be ordered from commercial sources such as Chemicon, Dharmacon, Panomics, or Qiagen.

As referred to herein, the term “siRNA” refers to a RNA or RNA analog comprising a gene or mRNA targeting region of between 15 and 35 nucleotides or nucleotide analogs, which RNA or RNA analog is capable of mediating RNA interference when the targeting region is paired with a homologous RNA sequence. With respect to the term “homologous”, it is understood that this term indicates that at least a portion of the RNA sequence bears sufficient homology to the target niRNA sequence to mediate RNA interference with respect to the target protein. It is preferred that the homologous portion of the RNA sequence bears 90% or greater sequence identity to the target mRNA sequence.

As referred to herein, a “gene or mRNA targeting region” refers to a sequence that is homologous to a gene or mRNA sequence corresponding to a portion of the protein of interest.

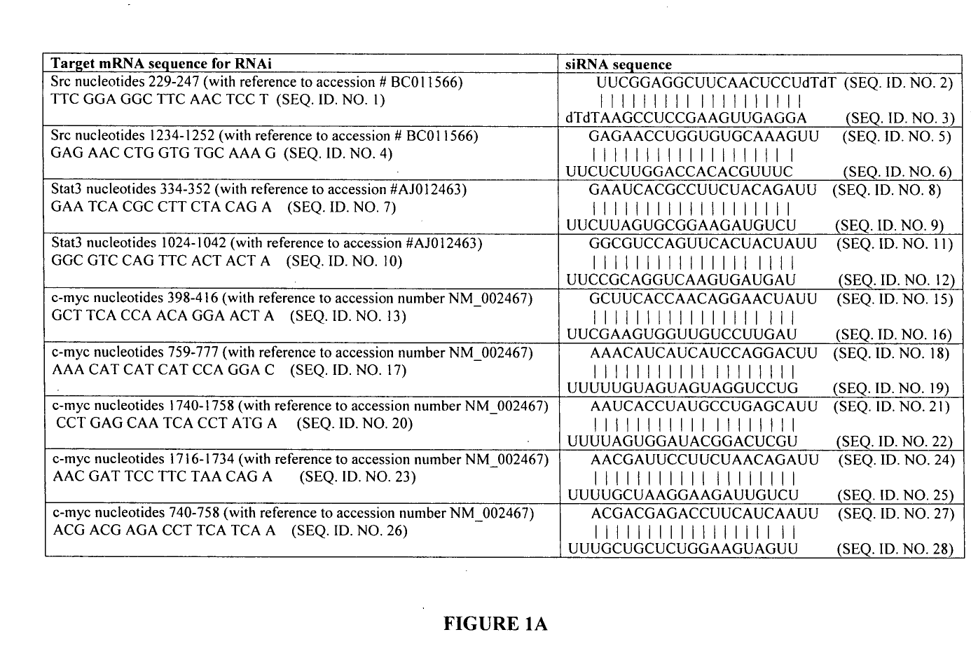

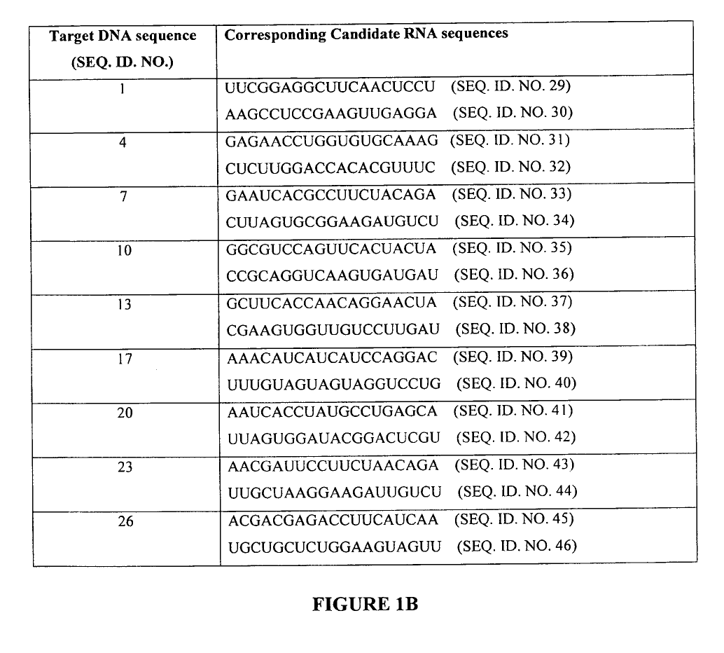

Examples of suitable siRNA sequences for use in accordance with the invention are shown in FIG. 1A. The target DNA sequences are also indicated in FIG. 1A with reference to the respective Genbank Accession numbers shown. These target DNA sequences were used to determine candidate RNA sequences as shown in FIG. 1B. These candidate RNA sequences were used to design the siRNA constructs shown in FIG. 1A, in which siRNA constructs are indicated for each of Src, Stat3, and cMyc. MAPK1, and Akt1 sequences used in the studies below were purchased from Upstate Biotechnology Inc. Other siRNA sequences may be designed in accordance with parameters known in the art, and additional effective siRNA sequences have also been previously disclosed (for example, for stat3: Lee et al, 2004, Prostate, Vol. 60, pp. 303-309; Ling and Arlinghaus, 2005; and for c-myc: Wang et al, 2005; Kabilova et al, 2004, Nucleosides Nucleotides Nucleic Acids, Vol. 23, pp. 867-872), which may also be used in accordance with the present invention.

With respect to Src siRNA sequence design, the preferred siRNA sequences are designed to target the N-terminal portion of the human c-src gene. This region is less conserved than the C-terminal portion of the gene, and has been proposed to encode Src-specific properties (Tanaka et al, 1987, Mol. Cell. Biol., Vol. 7, pp. 1978-1983). However, siRNA sequences targeting non N-terminal portions of the gene are also effective. Other general siRNA design parameters have been well-discussed in the literature (Elbashir, Harborth et al, 2001; Elbashir, Martinez et al, 2001) (Reynolds et al, 2004).

It is generally recommended that siRNA sequences should be between 18-23 nucleotides long, preferably with a 3′ dinucleotide overhang region, and a G-C content of 40-60% (Elbashir et al, 2001, Nature, Vol. 411, pp. 494-498; Elbashir et al, 2001, EMBO J., Vol. 20, pp. 6877-6888; Reynolds et al, 2004, Nat. Biotechnol., Vol. 22, pp. 326-330). Recent studies have shown that longer siRNA sequences of up to approximately 29 nucleotides (Kim et al, 2005, Nat. Biotechnol., Vol. 23, pp. 222-226; and Siolas et al, 2005, Nat. Biotechnol., Vol. 23, pp. 227-231) and hairpin siRNA sequences (Siolas et al, 2005) may be beneficial. In addition, modifications to the nucleotide backbone (Chiu et al, 2003, RNA, Vol. 9, pp. 1034-1048; Hamada et al, 2002, Antisense Nucleic Acid Drug Dev., Vol. 12, pp. 301-309; Chen et al, 2005, Drug Discov. Today, Vol. 10, pp. 587-593; Allerson et al, 2005, J. Med. Chem., Vol. 48, pp. 901-904; de Fougerolles et al, 2005, Methods Enzymol., VOl. 392, pp. 278-296; Morrissey et al, 2005, Hepatology, Vol. 41, pp. 1349-1356) as well as covalent/non-covalent molecules associated with the siRNA such as peptides (Davidson et al, 2004, J. Neurosci., Vol. 24, pp. 10040-10046), antibodies (Song et al, 2005, Nat. Biotechnol. Vol. 23, pp. 709-717), cholesterol (Soutschek et al, 2004, Nature, Vol. 432, pp. 173-178) may assist in the stability and/or delivery of the siRNA.

It is contemplated that the RNA sequences for use in accordance with the invention, including those RNA sequences specifically disclosed herein, may be modified to increase stability, or to provide further targeting or delivery optimization. For example, the RNA sequences for use in accordance with the invention may include additional nucleotides at either end of the sequence, may contain modified nucleotides, may be a single stranded RNA sequence folded into a hairpin loop RNA configuration, or may include functional or non-functional ligands, such as antibodies, tags, or other moieties as have been disclosed within the art (see above).

With respect to targeting a specific DNA or RNA sequence, it is contemplated that the specific DNA or RNA targets disclosed herein may be shortened or lengthened according to the Accession numbers provided, with corresponding homologous siRNA sequences designed accordingly. The specific siRNA sequences disclosed herein are effective embodiments with respect to the specific DNA and RNA targets disclosed, however, other siRNA sequences may also be designed based on the specific DNA and RNA targets disclosed herein, with direction from the present disclosure, and from the prior art.

Delivery of siRNA in vivo has been accomplished by liposome delivery (Flynn et al, 2004, J Inflamm (Lond), Vol 1(1), pp. 4) direct injection into tumors, iv injection (Song et al, 2003, Nature Medicine, Vol 9, pp. 347-351; Takahashi et al, 2005, J Control Release, e-published June 2, prior to print), adenoviral delivery (Tong et al, 2005, Curr Opin Mol Ther, Vol. 7(2), pp. 114-124), and by intravitreal injection (Tolentino et al, 2004, Retina, Vol. 24(1), pp. 132-138). It is believed to be within the skill of a person in the art to formulate and deliver the siRNA molecules and combination treatments in accordance with the invention.

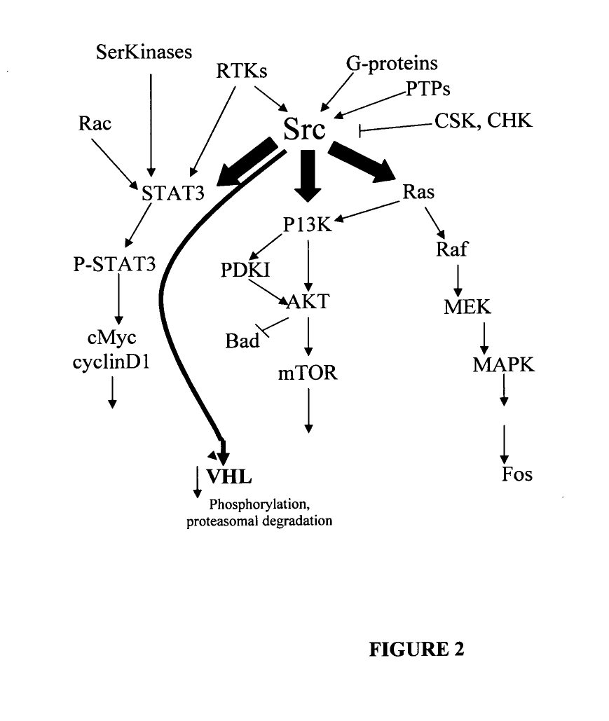

FIG. 2 depicts the possible convergence or interrelationship between Src and other signalling pathways of interest based on the studies discussed herein. Specifically, it has been previously suggested that the phosphorylation of Stat3 can be mediated by Src kinase, as well as certain other kinases. The results presented herein surprisingly indicate that targeted combination therapy to reduce expression of Src and Stat3 and/or c-Myc provide added benefit. This may occur because although Src is known to activate Stat3 by phosphorylation on Tyr 705, and to upregulate cMyc synthesis, there appear to be other parallel or interacting inputs or pathways that might also activate or influence the activity of Stat3 and/or Myc through a means that is not Src-dependent.

Proposed combination targets in accordance with certain embodiments of the invention include: Src plus inhibitors of Stat3, cMyc, Her2/Neu/EGFR2, EGFR1, VEGF, Flk1.KDR/VEGFR2, VEGFR1, FGFR, cMet, HGF/SF, FGF, P13K, PKB/Akt1/Akt2/Akt3, Ras, Raf, NFkB, MMP1, 2, 7, and 9. Due to the central role of Src, we propose that Src can be activated through upstream events involving virtually all tyrosine kinase growth factor receptors, and similarly, Src can activate numerous downstream pathways and events. It is contemplated herein that targeting Src with combined targeting of any other protein involved in a Src signaling pathway will provide an additive or synergistic effect in reducing cellular proliferation, and in cancer treatment.

Therapy in accordance with the above target combinations may be achieved by RNA interference, antibody-based therapy, drug targeting, or a combination thereof. For example, certain antibody-based therapies have been developed to reduce the effect of specific proteins involved in various diseases. In addition, drug therapies have been developed to interfere with certain intracellular signalling pathways. It is therefore believed to be within the skill of a person in the art to read the present disclosure and determine appropriate combination therapies based upon known RNA interference reagents, antibodies, and other targeted therapeutics.

Specifically, the use of siRNA to reduce the expression of Src alone and in combination with other signaling proteins provides specific inactivation and control over cell proliferation that is not possible with previous technologies or with previous reagents (for reference, Davies et al, 2000, Biochem J., Vol. 351, pp. 95-105).

Therefore, in accordance with the invention, a combination treatment regimen may be designed to significantly inhibit or prevent unwanted cellular proliferation or other properties contributing to oncogenesis.

### EXAMPLE 1

### Reduced Src Expression and Activity Following Transfection with Src siRNA

We initially examined the ability of a Src-specific siRNA to inhibit the expression of Src protein and suppress Src tyrosine kinase activity. A 21 nucleotide siRNA was designed that would target nucleotides 229-247 of Src (Genbank Accesssion BC011566), in a region in which the nucleotides encode a portion of the unique domain of the human Src protein (Tanaka et al., 1987). The highly metastatic human breast cancer cell line MDA-MB-435S was chosen to test the effectiveness of the siRNA because it has previously been demonstrated that Src activity was elevated and exhibited a higher kinase-specific activity in this cell line (Egan et al., 1999), and therefore might have an important role in maintaining the neoplastic phenotype of this cell line. This cell line can be induced to express breast differentiation-specific proteins and to secrete milk lipids as observed in other well-established breast cancer cell lines (Sellappan et al, 2004, Cancer Res., Vol. 64, pp. 2479-2485).

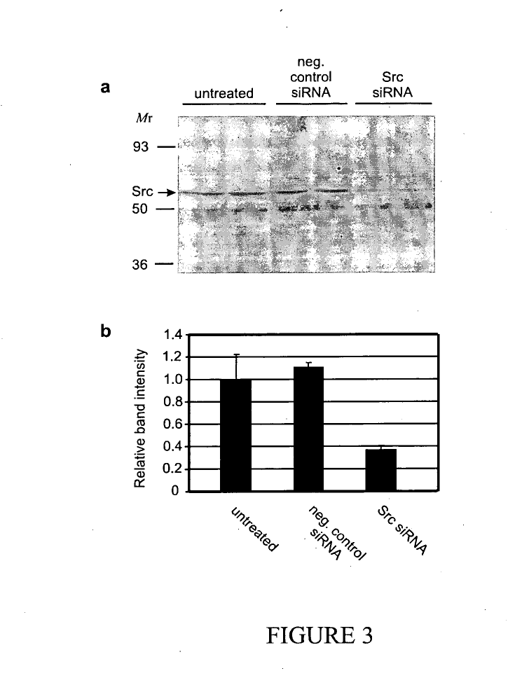

With reference to FIGS. 3A and B, Src siRNA caused a reduction in Src protein expression and activity. MDA-MB-435S cells were left untreated or transfected with non-targeting negative control siRNA or Src-targeting siRNA. 48 hours after transfection, cells for protein analysis were lysed. The extracts were either resolved by 9% SDS-PAGE, transferred to nitrocellulose, and probed with anti-Src antibody, or immunoprecipitated with anti-Src antibody and Protein G agarose. Src kinase activity was assayed utilizing a synthetic peptide substrate as previously described (Bjorge et al, 2000). The bands on the immunoblot were quantitated and the result is shown in FIG. 3b. Each data point is the mean of duplicate samples ±S.D. and is representative of at least 3 independent experiments.

When an siRNA directed against Src was transfected into MDA-MB-435S cells, typically, a 60-70% reduction in Src protein levels was observed 48 hours post-transfection relative to untransfected cells. Cells transfected with a non-targeting negative control siRNA showed no significant change in Src protein levels. This reduction was paralleled by a corresponding reduction in Src tyrosine kinase activity as measured by the phosphorylation of a synthetic substrate peptide. These results suggested that the Src siRNA was both capable of reducing the overall concentration of Src protein in the MDA-MB-435S cells as well as lowering the endogenous Src tyrosine kinase activity in these cells.

### EXAMPLE 2

### Src Suppression Affects the Anchorage-independent Growth of MDA-MB-435S Cells

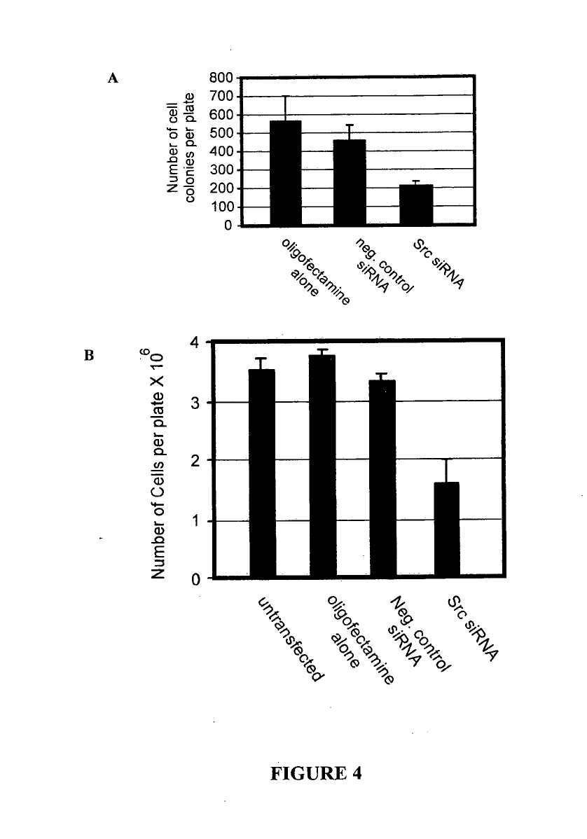

With reference to FIG. 4, Src siRNA causes a reduction in cell growth in soft agar and monolayer culture. MDA-MB-435S cells were left untreated or were transfected with non-targeting negative control siRNA or Src-targeting siRNA. Twenty-four hours after the transfection, cells for growth experiments were trypsinized and replated either into soft agar or into tissue culture dishes for monolayer growth experiments. The cells in soft agar were grown for 14 days. Each condition was assayed in triplicate and is representative of at least 3 independent experiments.

The ability of the Src siRNA to suppress anchorage-independent growth in soft agar was examined, which is a characteristic of transformed or cancer cells and is highly correlated with tumorigenicity. The Src-targeting siRNA was able to suppress MDA-MB-435S cell growth in soft agar by reducing the number of colonies in soft agar by 61% (FIG. 4a).

The MDA-MB-435S cells were also treated with Src siRNA and examined for alterations in anchorage-dependent cell growth rate in monolayer cultures. When MDA-MB-435S cells were treated with the Src siRNA, an approximate 50% reduction in cell number in comparison to untreated cells was observed 5 days post transfection (FIG. 4b). This effect was caused through the action of the Src siRNA because a non-targeting negative control siRNA was incapable of significantly reducing cell growth.

### EXAMPLE 3

### Targeted siRNA Treatment Reduces Expression of Various Signalling Molecules

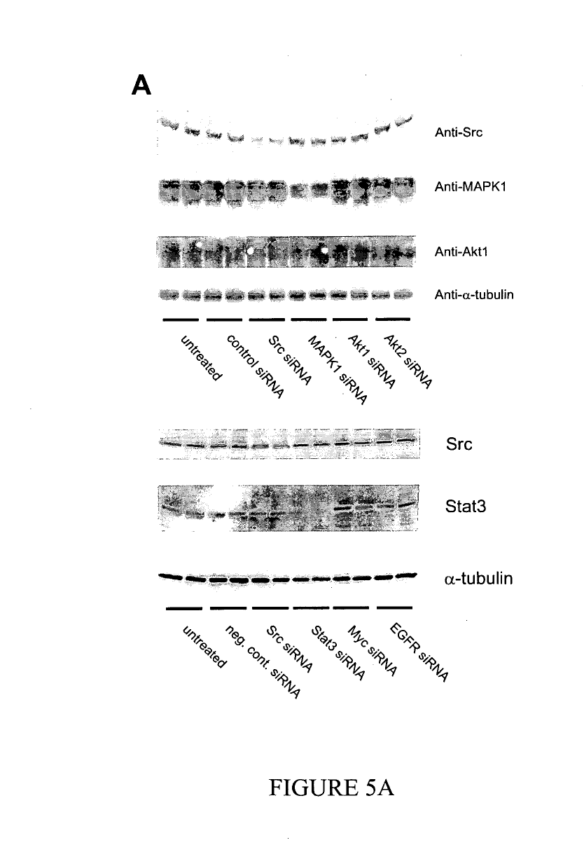

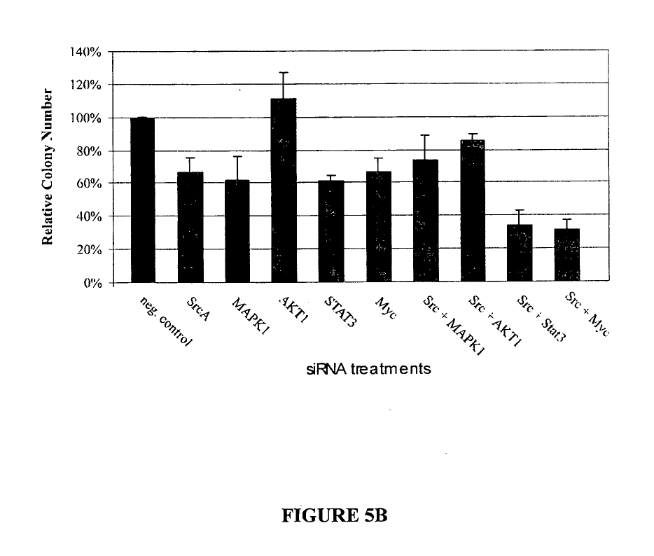

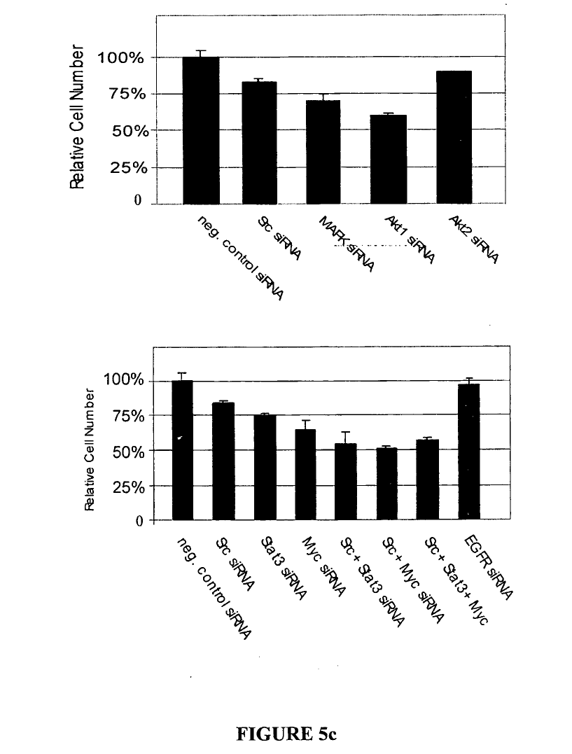

With reference to FIG. 5A through C, transfection of MDA-MB-435S cells with siRNA targeting various signaling molecules results in a reduced level of expression of their corresponding proteins, causing reductions in anchorage-independent and dependent growth. MDA-MB-435S cells in 12 well dishes were transfected with siRNA. 24 hrs post transfection, cells for growth experiments were plated into soft agar or into normal tissue culture dishes. Cells to be analysed by immunoblotting were lysed 60 hrs post transfection in RIPA buffer containing protease inhibitors and equal amounts of protein, and were subjected to SDS-PAGE. Following transfer to nitrocellulose, the samples were blotted with the corresponding antibodies. Duplicate samples of each cell extract were analyzed and results are shown in FIG. 5A. The cells in soft agar were grown for 14 days and colonies larger than approximately 50 cells were quantitated (results shown in FIG. 5B). The cells in monolayer culture were quantitated 4 days after plating (results shown in FIG. 5C). Each growth condition was assayed in triplicate and is representative of at least 3 independent experiments.

We examined if the cell growth effects we observed utilizing Src siRNA alone could be further augmented by combining the Src siRNA with siRNAs that target signaling pathways that are either downstream of Src or that interact with the Src signal transduction cascade (FIG. 2). siRNAs targeting proteins in several signaling pathways including MAPK1, Akt1, Myc, and Stat3 were examined for their ability to modify cell growth.

These siRNAs were initially examined for their ability to reduce their corresponding target proteins. Western blots carried out 60 hours post-transfection demonstrated that these siRNAs could significantly reduce protein expression levels of their corresponding proteins in whole cell extracts (FIG. 5a). The effects of the siRNAs were specific in that each siRNA had no significant effect on other proteins examined in the blots. The degree of reduction varied somewhat between the different siRNAs, but these siRNAs were capable of reducing protein levels by at least 60-95% (blots quantitated by phosphoimager, results not shown).

MDA-MB-435S cells were transfected with a single targeting siRNA or various siRNAs in combination with Src siRNA and the effects on growth in soft agar were examined. In contrast to the experimental protocols utilized in previous figures, cells were transfected only once. In this study, Src, MAPK1, Stat3, and Myc siRNA on their own were capable of causing a 30-40% reduction in the ability of the MDA-MB-435S cells to form colonies in soft agar (FIG. 5B). Combining Src siRNA with Stat3 or Myc siRNA had the maximal effect, resulting in a 65-70% reduction in colony number.

The ability of the siRNAs individually and in combination with Src siRNA to cause changes in cell growth in monolayer culture (see FIG. 5C) was observed. Cells were transfected, incubated for 4 days, and then quantitated. When cells were treated with the different individual siRNAs, we observed that the Src, MAPK1, Akt1, Stat3, and Myc siRNA reduced the cell number by 20% to 40%. When Src siRNA was combined with the other siRNAs, small additional reductions in monolayer cell growth were observed in combination with either Myc siRNA or Stat3 siRNA.

With reference to Examples 2 and 3, and to the results shown in FIGS. 4 and 5, siRNA-targeted inactivation of Src has substantial inhibitory effects (50-70% inhibition) on anchorage-independent growth (AIG) of colonies in semi-solid medium (a property that is highly correlated to tumorigenicity (Montesano et al, 1977, J. Natl. Cancer Inst., Vol. 59, pp. 1651-1658; San et al, 1979, Cancer Res., Vol. 39, pp. 1026-1034; Trainer et al, 1988, Int. J. Cancer, Vol. 41, pp. 287-296), partially through an increase in apoptosis. Lower degrees of inhibition of AIG were observed when other signaling elements (EGFR, Akt1, Akt2, cMyc, MAPK1[ERK2], Stat3) known or thought to be activated or upregulated by Src activity were targeted for inactivation. However, siRNA targeting of Src in pairwise combination with some of these elements produced a greater degree of inhibition of AIG (75-85%) than inactivation of Src alone.

### EXAMPLE 4

### Reduction of Tumor Formation in NOD/SCID Mice

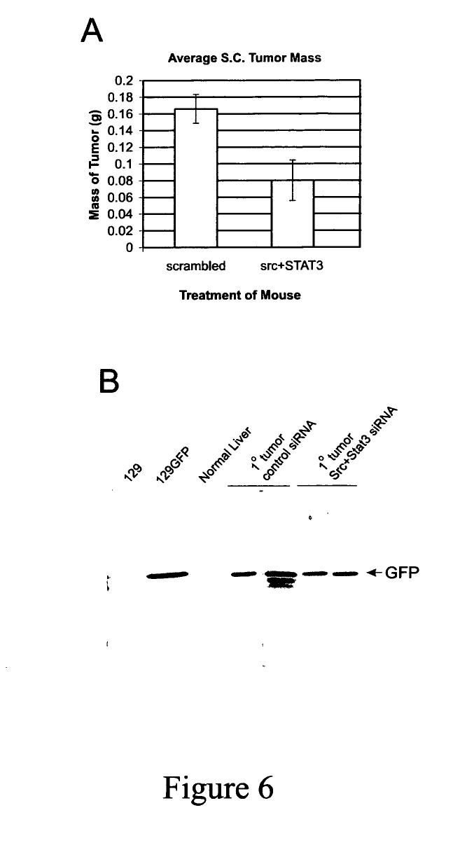

We next examined if the treatment of the MDA-MB-435S cells would affect their ability to form tumors in NOD/SCID mice. To facilitate the identification of the injected tumor cells, MDA-MB-435S cells were utilized that stably expressed GFP protein. 1×10⁶ siRNA treated cells (negative control or Src+Stat3 siRNA) were injected into the mammary fat pad of NOD/SCED mice and the mice were monitored for tumor growth. The mice were observed over a period of 7-9 weeks for the presence of tumors. During the post-injection period, the development of palpable tumors was monitored at the site of injection in all the mice. The mice underwent pathological examination between days 64-68, which revealed the presence of tumors at the site of injections. The tumors were excised, dissected free of normal tissue, and weighed (see results in FIG. 6A). A portion of each tumor was fixed in formaldehyde and sectioned. Slides of the sections were stained with hematoxylin and eosin or subjected to immunostaining with anti-GFP antibody. A portion of each tumor was homogenized with RIPA buffer and subjected to western blotting with anti-GFP antibodies (results in FIG. 6B).

The average mass of the tumors from the mice injected with cells treated with control siRNA was 0.167 g and were roughly twice the size of the tumors found in the mice treated with Src+Stat3 siRNA, where tumors averaged 0.080 g (FIG. 6A). Microscopic examination of sections from the tumors showed the presence of malignant cells in the tumor and the presence of the MDA-MB-435S cells was confirmed by immunostaining using antibody against GFP (results not shown). Western blotting for GFP in the isolated tumor specimens also showed the presence of GFP in the malignant cells and confirmed that they originated from the injected cells. These results demonstrated that treatment of the MDA-MB-435S cells with the Src+Stat3 siRNA was able to significantly reduce the ability of the cells to form tumors in the mice.

### EXAMPLE 5

### Src Suppression Increases Apoptosis in MDA-MB435S Cells

We were interested in whether the effects on cell growth might be at least partially explained by the ability of the Src siRNA to induce apoptosis in the transfected cells. Src has been previously described to have an anti-apoptotic activity by causing increased expression of the anti-apoptotic factor Bcl-XL in some cell types (Kami et al, 1999, Oncogene, Vol. 18(33), pp. 4654-4662). To examine this possibility, we utilized the TUNEL assay, which measures the appearance of free DNA ends that are the result of DNA fragmentation that occurs in the latter stages of apoptosis.

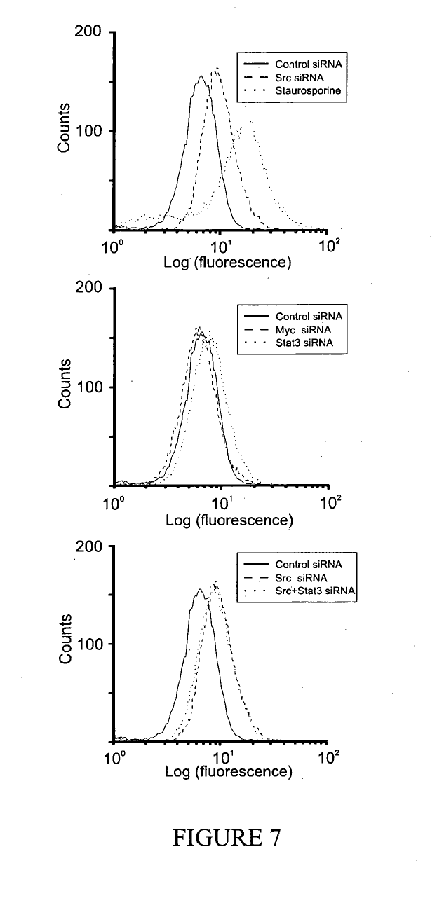

MDA-MB-435S cells in 6 well dishes were transfected with the corresponding siRNA or treated with 1 uM staurosporine. 48 hours post-transfection, any detached cells were combined with cells detached using trypsin and the cells were washed, fixed with paraformaldehyde, subjected to TUNEL assay, and analyzed by FACS. Results are expressed in FIG. 7A through C with log(fluorescence) on the horizontal axis and cell counts on the vertical axis and is representative of triplicate experiments.

Src siRNA was found to cause a significant increase in apoptosis in the MDA-MB-435S cells as evidenced by an increased incorporation of fluorescein-labeled dUTP into DNA of the treated cell population as examined by FACS analysis (FIG. 7A, upper panel, dashed line) as compared to cells treated with control siRNA (solid line). Staurosporine, a potent pro-apoptotic agent used as a control, caused the most dramatic increase in apoptotic cells (dotted line). Stat3 siRNA caused a smaller, but still significant shift in the cell population (FIG. 7B). Myc siRNA caused no significant change above what was observed with the negative control siRNA. Combining Src plus Stat3 siRNA was not additive or synergistic (FIG. 7C), as the cells had the same level of fluorescence as that observed with Src alone, suggesting the two inhibitors may share a similar pathway for inducing apoptosis.

Comments on Src siRNA

Suppression of Src using siRNA in the human breast cancer line MDA-MB-435S has a significant effect on the anchorage-independent growth characteristics of the cells. Src siRNA dramatically reduced the ability of the MDA-MB-435S cells to produce colonies in soft agar, an important hallmark of cell transformation, and to a lesser extent, reduced the growth rate of the MDA-MB-435S cell in monolayer cultures. In the NOD/SCID mouse tumor model, cells treated with Src siRNA also showed a strong reduction in the ability to form tumors at the primary site of implantation in the breast tissue. This suggests that even though the activation of Src activity may have occurred early on in the development of the neoplastic phenotype, it may still serve as a potential therapeutic target.

The effects of Src siRNA were further accentuated by combining it with siRNAs targeting proteins in related pathways. Further improvements in technology and in the stability of siRNA are likely to make this approach more efficient. The effects that were observed when utilizing only the Src siRNA are the consequence of specific inhibition of Src, as the siRNA methodology allows the specific targeting of Src, and none of the other Src family tyrosine kinases.

### EXAMPLE 6

### Effects of siRNA Treatment on Tumor Growth in Mice Injected with MDA-MD435S Breast Cancer Cells

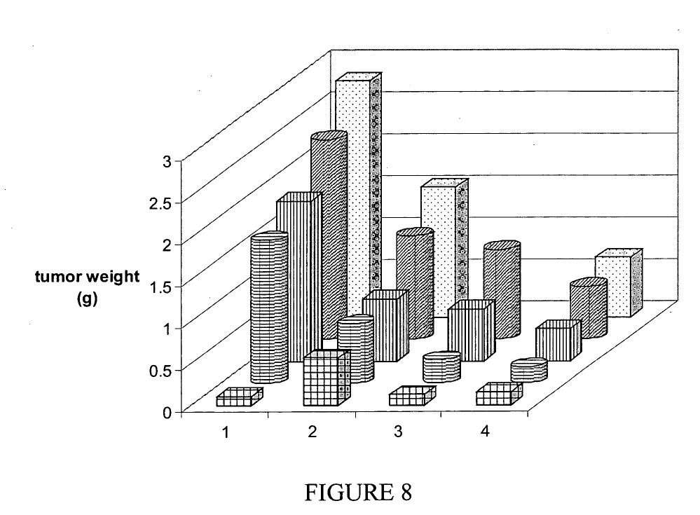

With reference to FIG. 8, four groups of mice were implanted with MDA-MD-435S cells and received injections of siRNA at the implantation site. Group 1 mice received MDA-MD-435S implantation MFP on day 0, and with scrambled siRNA on day1, and biweekly for approximately 120 days. Group 2 mice were implanted with MDA-MD-435S cells on day 0, and were injected with scrambled siRNA on day 1, and then biweekly for 6 week, whereupon the scrambled injections were replaced with Src siRNA plus stat3 siRNA injections biweekly until approximately day 120. Group 3 mice were implanted with MDA-MD-435S cells on day 0, and were injected with Src siRNA plus stat3 siRNA on day 1, and biweekly for approximately 120 days. Group 4 mice were implanted with MDA-MD-435S cells on day 0, and were injected with Src siRNA, stat3 siRNA, and c-myc siRNA on day 1, and biweekly for 120 days. As shown in FIG. 8, tumor weight was greater in Group 2 compared to Group 3, due to the delay in administration of siRNA by 6 weeks. The triple combination siRNA treatment (Src, Stat3, c-Myc) was associated with the lowest tumor weights. At least five mice were present in each experimental group.

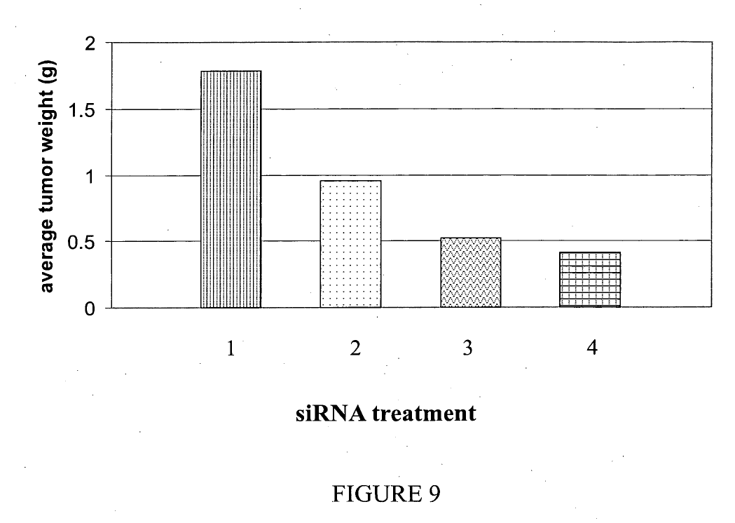

The average tumor weights for each group after 120 days are presented in FIG. 9, with the group receiving the scrambled siRNA sequences (Group 1) having the greatest average tumor weight, and the groups receiving the combined siRNA treatments beginning on day 1 having the lowest tumor weights.

These results suggest that early siRNA therapeutic intervention in breast cancer may reduce tumor weight by inhibiting cellular signalling of proliferative pathways. In addition, the results suggest that combination siRNA treatment may be effective in effecting such inhibition.

### EXAMPLE 7

### Effects of siRNA Treatment on Tumor Growth in Mice Injected with SW480 Colon Cancer Cells

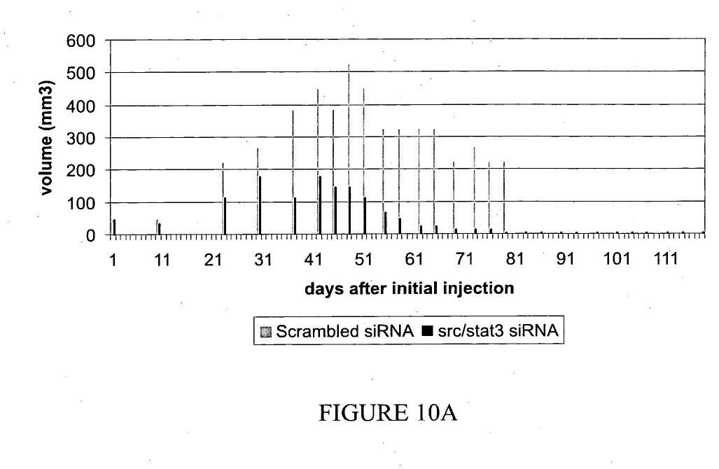

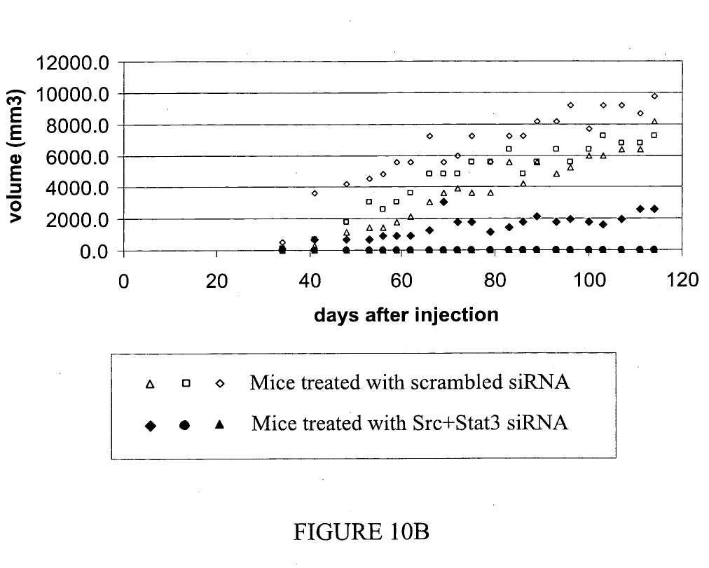

With reference to FIG. 10A, mice were injected with SW480 colon cancer cell line on Day 0, and scrambled or combined Src and Stat3 siRNA was injected daily thereafter for approximately 80 days. As shown in FIG. 8, the tumor volume in the mice receiving the combined siRNA treatment was greatly reduced compared to the mice receiving scrambled siRNA treratment.

Similarly, with reference to FIG. 10B, mice were injected with SW480 colon cancer cells which had been transfected with either scrambled siRNA or combined Src plus stat3 siRNA. Tumor volume in the group receiving combined siRNA treatment had reduced tumor volume after 110 days when compared with the control group.

Methods

Single-stranded forms of siRNA were first deprotected, purified by ethanol precipitation, quantitated by absorbance at 260 nm, and annealed according to the manufacturer's instructions. The double-stranded unprotected forms were utilized without any further modifications. The double stranded siRNA were all synthesized to possess 2 nucleotide 3′-terminal overhangs.

327 anti-Src antibody (Lipsich et al, 1983, J. Virol., Vol. 48, pp. 352-360) was a kind gift from J. Brugge. 4G10 antiphosphotyrosine antibody was a kind gift from S. Robbins. Ab-1 anti-α-tubulin antibody was from Oncogene Research Products. Anti-GFP antibody was from Santa Cruz. Antibodies against Mapk, Myc, Stat3, Akt1, and Akt2 were from Upstate Biotech. Inc. H9B4 antibody against the EGFR has been previously described (Bjorge and Kudlow, 1987, J. Biol. Chem., Vol. 262, Pp. 6615-6622).

MDA-MB-435S breast cancer cells were obtained from American Type Culture Collection and were maintained in Dulbecco's modified Eagle's medium containing 10% fetal bovine serum. The cells were seeded 24 hour prior to transfection in 24 well tissue culture dishes and were at 70% confluency on the day of transfection. Transfections were carried out using oligofectamine (Invitrogen) in OptiMEM medium in the absence of serum according to the manufacturer's protocol, with the various siRNAs at a final concentration of 200 nM. 24 hours after the first transfection, the cells were detached using trypsin, and replated in 12 well tissue culture dishes. The cells were retransfected the following day to ensure a high percentage of the cells became transfected (using this protocol, we calculate that greater than 90% of the cells were transfected, with little or no observed cytotoxicity).

Cells whose proteins were to be analyzed by immunoblotting were lysed using RIPA buffer (Bjorge et al., 2000) on ice, the extracts were clarified by centrifugation for 10 minutes at 10,000×g, and the supernatants removed. The protein concentration of the supernatants was determined using Bradford protein reagent (Biorad), and equal amounts of cell extract (approximately 70 μg) was subjected to SDS-PAGE. The proteins were transferred to nitrocellulose and probed with primary antibody, followed by horseradish peroxidase-conjugated secondary anti-IgG antibody. Detection of the bands was carried out using ECL plus detection reagent (Amersham). Images of the blot were obtained by exposure with X-OMat AR film or scanning the filter using a Storm 860 PhosphorImager (Molecular Dynamics). The bands were quantitated using ImageQuant software.

Cells to be grown as monolayers were replated into 10 cm tissue culture dishes in 2.5% fetal bovine serum/2.5% calf serum in Dulbecco's modified Eagle's medium at a density of 1.5×10⁵ cells/dish. The cells had their medium changed once 3 days after plating and the cell number was quantitated following detachment using trypsin using a Beckman Z1 Cell and Particle Counter 5 days after plating. Cells to be assayed for their ability to grow in soft agar were suspended in 3.0 ml of 0.30% agar in Dulbecco's Modified Eagle's medium containing 10% fetal bovine serum at a density of 2.35×10⁴ cells/dish and plated on a layer of prehardened 0.5% agar in the same medium as above. The cells were overlayed with fresh medium containing 0.3% agarose every 4 days and the colonies larger than approximately 50 cells were quantitated using a microscope after 14 days. Images of the plates were made using a Microtek scanner.

Cells to be injected into mice were transfected on two consecutive days with either the negative control siRNA or Src and Stat3 siRNAs. The cells were then detached from the dishes using trypsin and washed twice and resuspended in phenol red-free DMEM/PBS (50/50) at a concentration of 1×10⁶ cells/100 μl. 1×10⁶ cells were then injected into the mammary fat pad of 5 week old NOD/SCID mice (Jackson Laboratory). The mice were observed over a period of 7-9 weeks for the presence of tumors.

Cells to be assayed were trypsinized, washed twice with PBS, followed by fixation for 20 min. in 1.5% paraformaldehyde in PBS at 4° C. with gentle mixing. The cells were then washed twice with PBS, suspended in 70% ethanol at 4° C., and stored at −20° C. until assayed. When assayed, the cells were washed twice with PBS and then resuspended in 50 μl of Tunel assay buffer consisting of 100 mM potassium cacodylate (pH 7.2), 2 mM CoCl₂, 0.2 mM dithiotheitol, 3.3 nM fluorescein-conjugated dUTP, 13 nM DATP and 11 units of terminal deoxynucleotidyl transferase for 90 minutes at 37° C. The cells were then washed twice with PBS and analyzed by FACS sorting.

With respect to the mouse intratumoral injections of siRNA, 14 ug of siRNA in 50 ul of Optimem medium was mixed with 50 ul of Oligofectamine Reagent/Optimem medium according to the manufacturers instructions protocol. The prepared siRNA/Oligofectamine complexes were then injected into 2 sites within the tumor. Injections were carried out twice weekly during the duration of the experiment.

All references cited above are incorporated by reference to the same extent as if each reference had been incorporated by reference individually in its entirety. The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. An isolated RNA molecule comprising a nucleic acid molecule hybridized to a second nucleic acid molecule, the first nucleic acid molecule selected from the group consisting of:

| | |
|---|---|
| 5′-UUCGGAGGCUUCAACUCCU-3′; | (SEQ. ID. NO. 29) |
| 3′-AAGCGUCCGAAGUUGAGGA-5′; | (SEQ. ID. NO. 30) |
| 5′-GAGAACCUGGUGUGCAAAG-3′; | (SEQ. ID. NO. 31) |
| 3′-GUCUUGGACCACACGUUUC-5′; | (SEQ. ID. NO. 32) |
| 5′-GAAUCAGGCCUUCUACAGA-3′; | (SEQ. ID. NO. 33) |
| 3′-CUUAGUGCGGAAGAUGUCU-5′; | (SEQ. ID. NO. 34) |
| 5′-GGCGUGCAGUUCACUACUA-3′; | (SEQ. ID. NO. 35) |
| 3′-CCGGAGGUCAAGUGAUGAU-5′; | (SEQ. ID. NO. 36) |
| 5′-GCUUGACGAACAGGAACUA-3′; | (SEQ. ID. NO. 37) |
| 3′-CGAAGUGGUUGUCCUUGAU-5′; | (SEQ. ID. NO. 38) |
| 5′-AAACAUCAUCAUCCAGGAC-3′; | (SEQ. ID. NO. 39) |
| 3′-UUUGUAGUAGUAGGUCCUG-5′; | (SEQ. ID. NO. 40) |
| 5′-AAUCACCUAUGCCUGAGCA-3′; | (SEQ. ID. NO. 41) |
| 3′-UUAGUGGAUACGGACUCGU-5′; | (SEQ. ID. NO. 42) |
| 5′-AACGAUUCCUUGUAACAGA-3′; | (SEQ. ID. NO. 43) |
| 3′-UUGGUAAGGAAGAUUGUGU-5′; | (SEQ. ID. NO. 44) |
| 5′-ACGACGAGACCUUCAUCAA-3′; | (SEQ. ID. NO. 45) |
| and | |
| 3′-UGCUGGUCUGGAAGUAGUU-5′. | (SEQ. ID. NO. 46) |

2. The isolated RNA molecule of claim 1, wherein the RNA molecule further comprises additional nucleotides, functional groups, antibodies, or other ligands appended directly or indirectly to the RNA molecule.

3. A method for reducing cellular proliferation in vivo, comprising the step of administering at least one molecule in accordance with claim 1 to a cell, tissue, or mammal.

4. A method for inhibiting at least one intracellular signalling pathway comprising the step of administering at least one molecule in accordance with claim 1 to a cell, tissue, or mammal.

5. An siRNA for reducing expression of a protein involved in cellular proliferative signalling, the siRNA selected from the group consisting of:

| | |
|---|---|
| 5′-UUCGGAGGCUUCAACUCCUdTdT-3′ | (SEQ. ID. NO. 2) |
| hybridized to | |
| 3′-dTdTAAGCCUCCGAAGUUGAGGA-5′; | (SEQ. ID. NO. 3) |
| 5′-GAGAACCUGGUGUGCAAAGUU-3′ | (SEQ. ID. NO. 5) |
| hybridized to | |
| 3′-UUCUCUUGGAGCACACGUUUC-5′; | (SEQ. ID. NO. 6) |
| 5′-GAAUCACGCCUUCUAGAGAUU-3′ | (SEQ. ID. NO. 8) |
| hybridized to | |
| 3′-UUCUUAGUGCGGAAGAUGUCU-5′; | (SEQ. ID. NO. 9) |
| 5′-GGCGUCCAGUUCACUACUAUU-3′ | (SEQ. ID. NO. 11) |
| hybridized to | |
| 3′-UUCGGCAGGUCAAGUGAUGAU-5′; | (SEQ. ID. NO. 12) |
| 5′-GCUUCACCAACAGGAACUAUU-3′ | (SEQ. ID. NO. 15) |
| hybridized to | |
| 3′-UUCGAAGUGGUUGUCCUUGAU-5′; | (SEQ. ID. NO. 16) |
| 5′-AAACAUCAUCAUCCAGGACUU-3′ | (SEQ. ID. NO. 18) |
| hybridized to | |
| 3′-UUUUUGUAGUAGUAGGUCGUG-5′; | (SEQ. ID. NO. 19) |
| 5′-AAUCACCUAUGCCUGAGCAUU-3′ | (SEQ. ID. NO. 21) |
| hybridized to | |
| 3′-UUUUAGUGGAUACGGACUCGU-5′; | (SEQ. ID. NO. 22) |
| 5′-AACGAUUCGUUCUAACAGAUU-3′ | (SEQ. ID. NO. 24) |
| hybridized to | |
| 3′-UUUUGCUAAGGAAGAUUGUCU-5′; | (SEQ. ID. NO. 25) |
| and | |
| 5′-ACGACGAGACCUUCAUCAAUU-3′ | (SEQ. ID. NO. 27) |
| hybridized to | |
| 3′-UUUGCUGCUCUGGAAGUAGUU-5′. | (SEQ. ID. NO. 28) |

6. The siRNA of claim 5, wherein one or both strands of RNA further comprise(s) additional nucleotides, functional groups, antibodies, or other ligands appended directly or indirectly to the RNA strand(s).

7. A double-stranded RNA molecule that inhibits Src expression in a cell, wherein one of the RNA strands is homologous to at least a portion of a Src DNA or RNA molecule.

8. The RNA molecule of claim 7 wherein the portion of the Src DNA or RNA molecule is a conserved portion of the Src DNA or RNA molecule.

9. The RNA molecule of claim 7 that is between 15-35 base pairs in length.

10. The double-stranded RNA molecule of claim 7, wherein the RNA molecule is a hairpin RNA sequence.

11. The double-stranded RNA molecule of claim 7, wherein the double-stranded RNA molecule further comprises additional nucleotides, functional groups, antibodies, or other ligands appended to the RNA molecule.

12. A method for reducing cellular proliferation in vivo comprising the step of administering the RNA molecule as in claim 7 to a cell or mammal.

13. A method for inhibiting at least one intracellular signalling pathway comprising the step of administering the RNA molecule as in claim 7 to a cell or mammal.

14. A method for the treatment of cancer comprising administering an RNA molecule as in claim 7 to a patient in need thereof.

15. The method as in claim 14 wherein the cancer is breast or colon cancer.

16. A method to reduce cell proliferation comprising administering to a cell an effective amount of a reagent that reduces Src expression; and administering to a cell an effective amount of an additional reagent that reduces activity of at least one non-Src protein, wherein the non-Src protein is a protein involved in intracellular proliferative signalling.

17. A method in accordance with claim 16 wherein the reagent that reduces Src expression is a double stranded RNA molecule.

18. A method in accordance with claim 17 wherein the additional reagent is a double stranded RNA molecule, an antibody, or a targeted drug therapy reagent.

19. The method as in claim 16 wherein the protein involved in intracellular proliferative signalling is Stat3 or cMyc.


SEQUENCE LISTING (46 sequences)

SEQ ID NO: 1 - DNA, 19 bp, Homo sapiens
ttcggaggcttcaactcct

SEQ ID NO: 2 - DNA, 21 bp, Artificial Sequence
  siRNA construct for reducing expression of a protein involved in cellular proliferative signaling
  misc (1)..(21): This is a combined DNA/RNA sequence.
uucggaggcuucaacuccutt

SEQ ID NO: 3 - DNA, 21 bp, Artificial Sequence
  siRNA construct for reducing expression of a protein involved in cellular proliferative signaling
  misc (1)..(21): This is a combined DNA/RNA sequence.
aggaguugaagccuccgaatt

SEQ ID NO: 4 - DNA, 19 bp, Homo sapiens
gagaacctggtgtgcaaag

SEQ ID NO: 5 - RNA, 21 nt, Artificial Sequence
  siRNA construct for reducing expression of a protein involved in cellular proliferative signaling
gagaaccuggugugcaaaguu

SEQ ID NO: 6 - RNA, 21 nt, Artificial Sequence
  siRNA construct for reducing expression of a protein involved in cellular proliferative signaling
cuuugcacaccagguucucuu

SEQ ID NO: 7 - DNA, 19 bp, Homo sapiens
gaatcacgccttctacaga

SEQ ID NO: 8 - RNA, 21 nt, Artificial Sequence
  siRNA construct for reducing expression of a protein involved in cellular proliferative signaling
gaaucacgccuucuacagauu

SEQ ID NO: 9 - RNA, 21 nt, Artificial Sequence
  siRNA construct for reducing expression of a protein involved in cellular proliferative signaling
ucuguagaaggcgugauucuu

SEQ ID NO: 10 - DNA, 19 bp, Homo sapiens
ggcgtccagttcactacta

SEQ ID NO: 11 - RNA, 21 nt, Artificial Sequence
  siRNA construct for reducing expression of a protein involved in cellular proliferative signaling
ggcguccaguucacuacuauu

SEQ ID NO: 12 - RNA, 21 nt, Artificial Sequence
  siRNA construct for reducing expression of a protein involved in cellular proliferative signaling
uaguagugaacuggacgccuu

SEQ ID NO: 13 - DNA, 19 bp, Homo sapiens
gcttcaccaacaggaacta

SEQ ID NO: 14 - skipped

SEQ ID NO: 15 - RNA, 21 nt, Artificial Sequence
  siRNA construct for reducing expression of a protein involved in cellular proliferative signaling
gcuucaccaacaggaacuauu

SEQ ID NO: 16 - RNA, 21 nt, Artificial Sequence
  siRNA construct for reducing expression of a protein involved in cellular proliferative signaling
uaguuccuguuggugaagcuu

SEQ ID NO: 17 - DNA, 19 bp, Homo sapiens
aaacatcatcatccaggac

SEQ ID NO: 18 - RNA, 21 nt, Artificial Sequence
  siRNA construct for reducing expression of a protein involved in cellular proliferative signaling
aaacaucaucauccaggacuu

SEQ ID NO: 19 - RNA, 21 nt, Artificial Sequence
  siRNA construct for reducing expression of a protein involved in cellular proliferative signaling
guccuggaugaugauguuuuu

SEQ ID NO: 20 - DNA, 19 bp, Homo sapiens
cctgagcaatcacctatga

SEQ ID NO: 21 - RNA, 21 nt, Artificial Sequence
  siRNA construct for reducing expression of a protein involved in cellular proliferative signaling
aaucaccuaugccugagcauu

SEQ ID NO: 22 - RNA, 21 nt, Artificial Sequence
  siRNA construct for reducing expression of a protein involved in cellular proliferative signaling
ugcucaggcauaggugauuuu

SEQ ID NO: 23 - DNA, 19 bp, Homo sapiens
aacgattccttctaacaga

SEQ ID NO: 24 - RNA, 21 nt, Artificial Sequence
  siRNA construct for reducing expression of a protein involved in cellular proliferative signaling
aacgauuccuucuaacagauu

SEQ ID NO: 25 - RNA, 21 nt, Artificial Sequence
  siRNA construct for reducing expression of a protein involved in cellular proliferative signaling
ucuguuagaaggaaucguuuu

SEQ ID NO: 26 - DNA, 19 bp, Homo sapiens
acgacgagaccttcatcaa

SEQ ID NO: 27 - RNA, 21 nt, Artificial Sequence
  siRNA construct for reducing expression of a protein involved in cellular proliferative signaling
acgacgagaccuucaucaauu

SEQ ID NO: 28 - RNA, 21 nt, Artificial Sequence
  siRNA construct for reducing expression of a protein involved in cellular proliferative signaling
uugaugaaggucucgucguuu

SEQ ID NO: 29 - RNA, 19 nt, Artificial Sequence
  Candidate RNA sequence corresponding to target DNA sequence SEQ ID NO: 1
uucggaggcuucaacuccu

SEQ ID NO: 30 - RNA, 19 nt, Artificial Sequence
  Candidate RNA sequence corresponding to target DNA sequence SEQ ID NO: 1
aggaguugaagccuccgaa

SEQ ID NO: 31 - RNA, 19 nt, Artificial Sequence
  Candidate RNA sequence corresponding to target DNA sequence SEQ ID NO: 4
gagaaccuggugugcaaag

SEQ ID NO: 32 - RNA, 19 nt, Artificial Sequence
  Candidate RNA sequence corresponding to target DNA sequence SEQ ID NO: 4
cuuugcacaccagguucuc

SEQ ID NO: 33 - RNA, 19 nt, Artificial Sequence
  Candidate RNA sequence corresponding to target DNA sequence SEQ ID NO: 7
gaaucacgccuucuacaga

SEQ ID NO: 34 - RNA, 19 nt, Artificial Sequence
  Candidate RNA sequence corresponding to target DNA sequence SEQ ID NO: 7
ucuguagaaggcgugauuc

SEQ ID NO: 35 - RNA, 19 nt, Artificial Sequence
  Candidate RNA sequence corresponding to target DNA sequence SEQ ID NO: 10
ggcguccaguucacuacua

SEQ ID NO: 36 - RNA, 19 nt, Artificial Sequence
  Candidate RNA sequence corresponding to target DNA sequence SEQ ID NO: 10
uaguagugaacuggacgcc

SEQ ID NO: 37 - RNA, 19 nt, Artificial Sequence
  Candidate RNA sequence corresponding to target DNA sequence SEQ ID NO: 13
gcuucaccaacaggaacua

SEQ ID NO: 38 - RNA, 19 nt, Artificial Sequence
  Candidate RNA sequence corresponding to target DNA sequence SEQ ID NO: 13
uaguuccuguuggugaagc

SEQ ID NO: 39 - RNA, 19 nt, Artificial Sequence
  Candidate RNA sequence corresponding to target DNA sequence SEQ ID NO: 17
aaacaucaucauccaggac

SEQ ID NO: 40 - RNA, 19 nt, Artificial Sequence
  Candidate RNA sequence corresponding to target DNA sequence SEQ ID NO: 17
guccuggaugaugauguuu

SEQ ID NO: 41 - RNA, 19 nt, Artificial Sequence
  Candidate RNA sequence corresponding to target DNA sequence SEQ ID NO: 20
aaucaccuaugccugagca

SEQ ID NO: 42 - RNA, 19 nt, Artificial Sequence
  Candidate RNA sequence corresponding to target DNA sequence SEQ ID NO: 20
ugcucaggcauaggugauu

SEQ ID NO: 43 - RNA, 19 nt, Artificial Sequence
  Candidate RNA sequence corresponding to target DNA sequence SEQ ID NO: 23
aacgauuccuucuaacaga

SEQ ID NO: 44 - RNA, 19 nt, Artificial Sequence
  Candidate RNA sequence corresponding to target DNA sequence SEQ ID NO: 23
ucuguuagaaggaaucguu

SEQ ID NO: 45 - RNA, 19 nt, Artificial Sequence
  Candidate RNA sequence corresponding to target DNA sequence SEQ ID NO: 26
acgacgagaccuucaucaa

SEQ ID NO: 46 - RNA, 19 nt, Artificial Sequence
  Candidate RNA sequence corresponding to target DNA sequence SEQ ID NO: 26
uugaugaaggucucgucgu